(19) United States
(12) Patent Application Publication
(10) Pub. No.: US 2018/0140564 A1
(43) Pub. Date: May 24, 2018

(54) METHODS AND SYSTEMS FOR TREATING OVERWEIGHT INDIVIDUALS

(71) Applicant: LiveLight LLC, Palo Alto, CA (US)

(72) Inventors: Tadmor SHALON, Palo Alto, CA (US); Bradford RABIN, Palo Alto, CA (US)

(73) Assignee: LiveLight LLC, Palo Alto, CA (US)

(21) Appl. No.: 15/877,029

(22) Filed: Jan. 22, 2018

Related U.S. Application Data
(63) Continuation of application No. 14/827,207, filed on Aug. 14, 2015.
Application No. 14/827,207 is a continuation of application No. PCT/US2014/019482, filed on Feb. 28, 2014.
(60) Provisional application No. 61/770,838, filed on Feb. 28, 2013.

(51) Int. Cl. A61K 31/137; A61J 1/03

(52) U.S. Cl. CPC A61K 31/137; A61J 1/035

(57) ABSTRACT

A system for inducing weight loss in an individual includes a collection having plurality of dose units therein. Each dose unit includes a first dose of phentermine or a second dose of phentermine. The first dose is greater than the second dose, and the collection includes at least one first dose and at least one second dose.

### CROSS-REFERENCE TO RELATED APPLICATIONS

This application is a continuation of U.S. patent application Ser. No. 14/827,207 filed Aug. 14, 2015, which is a continuation of PCT International Application No. PCT/US2014/019482 filed Feb. 28, 2014, which claims priority to U.S. Provisional Patent Application No. 61/770,838 filed Feb. 28, 2013, each of which is incorporated by reference herein in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Various drugs and drug administration, doses, and dosing schedules have been used to treat obesity, but all have proven inadequate because they quickly lose their anorexiant effect due to patient buildup of tolerance and because they tend to have significant adverse effects.

Phentermine (.alpha.-dimethyl-.beta.-phenylethylamine) has been used as oral monotherapy for obesity since about 1970. Phentermine acts on the cerebral appetite center to reduce appetite. It is effective when given continuously for about two weeks, but then quickly loses effect. For this reason, the FDA does not approve continuous administration of therapeutic doses of phentermine for periods of time beyond twelve weeks.

Moreover, phentermine monotherapy is associated with important adverse effects, including insomnia and nervousness, resulting in reduced efficacy due to patient non-compliance. To avoid some of these side effects, previous studies looked into intermittent therapy by alternating phentermine and placebo over the trial period on a monthly or bimonthly basis. For example, Munro et al., “Comparison of Continuous and Intermittent Anorectic Therapy in Obesity,” Brit Med. J., 1968, pages 352-354, demonstrated that patients on a 12-week treatment with weeks 4 through 8 on placebo lost as much weigh on average as the group that received phentermine for all 12 weeks. Further, Truant et al., “Phentermine Resin as an Adjunct in Medical Weight Reduction: A Controlled, Randomized, Double-Blind Prospective Study,” Curr Ther Res Clin Exp., 1972, pages 726-738, demonstrated that patients who received phentermine for twelve weeks lost 18.8 lb while those who received phentermine for the same period except for placebo on weeks 4, 8, and 12 lost nearly as much weight (16.6 lb).

FIGS. 1A and 1B shows the results of various published clinical studies of phentermine.

Studies using phentermine alone or phentermine mixed with a placebo have reported significant patient dropout rates in a typical 8-12 weeks phentermine clinical trial (Lucey et al “Chlorphentermine, A New ‘Appetite Suppressant,’ A Cross-Over Double-Blind Trial,” Ulster Med. J., 1962, pages 181-184 reports a 66% dropout rate, Kim et al., “Effects on Weight Reduction and Safety of Short-Term Phentermine Administration in Korean Obese People,” Yonsei Med J., 2006 Vol. 47, No. 5, pages 614-625 reports a 47% dropout rate, Truant reports a 66% dropout rate, Munro reports a 41% dropout rate, and Langlois et al., “A Double-Blind Clinical Evaluation of the Safety and Efficacy of Phentermine Hydrochloride (Fastin) in the Treatment of Exogenous Obesity,” Curr Ther Res Clin Exp., 1974, Vol. 16, No. 4, pages 289-296 reports a 41% dropout rate). For broad clinical adoption, the intent to treat weight loss, e.g., the average weight loss of all the patients that were enrolled in the study, is the key measure. Referring to Table 1A, the intent to treat weight loss would be reduced to 9.0 lb for the patients receiving phentermine and 3.5 lb for those on placebo if the individuals who dropped out and did not lose any net weight were included in the results.

As a result of the drawbacks associated with phentermine, clinicians are reluctant to prescribe the drug for these reasons. For example, in the United States in 1992, less than 500,000 phentermine prescriptions were dispensed even though there were 60,000,000 people in the U.S. in need of medical therapy for obesity. However, phentermine remains a promising medication for inducing weight lots if used properly.

Thus, it would be highly advantageous to have a phentermine treatment regimen devoid of the above limitations while effectively reducing a patient's weight.

### BRIEF SUMMARY OF THE INVENTION

The present invention successfully addresses the shortcomings of the presently known configurations by providing a phentermine weight loss treatment regimen which traverses the loss-of-effect and side effect limitations typically associated with continuous phentermine therapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

In general, in one embodiment, a system for inducing weight loss in an individual includes a collection consisting of a one week supply of daily dose units. Each dose unit of the collection includes a first dose of phentermine or a second dose of phentermine. The first dose is greater than the second dose, and the collection includes at least one first dose and at least one second dose.

This and other embodiments can include one or more of the following features. All of the dose units can appear identical. The second dose can include 0 mg of phentermine. The second dose can include less than 4 mg of phentermine. The first dose can have at least 5 mg more phentermine than the second dose. The first dose can have at least 7.5 mg more phentermine than the second dose. The first dose can include 5-50 mg of phentermine. The first dose can include 7.5-35 mg of phentermine. Each collection can include at least two second doses. The dose units can be pills or capsules. The collection can be a pack, such as a blister pack. The dose units can be numbered. The dose units can be designated by days of the week.

In general, in one embodiment, a system for inducing weight loss in an individual includes a pack having plurality of dose units therein. Each dose unit includes a first dose of phentermine or a second dose of phentermine. The first dose is greater than the second dose, and the pack includes at least one first dose and at least one second dose.

This and other embodiments can include one or more of the following features. All of the dose units can appear identical. The second dose can include 0 mg of phentermine. The second dose can include less than 4 mg of phentermine. The first dose can have at least 5 mg more phentermine than the second dose. The first dose can have at least 7.5 mg more phentermine than the second dose. The first dose can include 5-50 mg of phentermine. The first dose can include 7.5-35 mg of phentermine. The pack can include at least two second doses. The pack can include less than a monthly supply of dose units. The dose units can be daily dose units. The pack can include at least a one-week supply of dose units. The dose units can be pills or capsules. The pack can be a blister pack. The dose units can be numbered. The dose units can be designated by days of the week.

In general, in one embodiment, a system for inducing weight loss in an individual includes a collection having plurality of dose units therein. Each dose unit includes a first dose unit of a medication or a second dose unit of medication. The first dose is greater than the second dose, and at least 30% of the dose units are second doses.

This and other embodiments can include one or more of the following features. Less than 90% of the dose units can be second doses. Each of the dose units can appear identical. There can be seven dose units in the collection. An order of taking the dose units can be indicated on the collection. The second dose can include 0 mg of phentermine. The second dose can include less than 4 mg of phentermine. The first dose can have at least 5 mg more phentermine than the second dose. The first dose can have at least 7.5 mg more phentermine than the second dose. The first dose can include 5-50 mg of phentermine. The first dose can include 7.5-35 mg of phentermine.

In general, in one embodiment, a method of treating a patient includes: (1) providing a first plurality of dose units of a medication to a patient according to a dose regimen; (2) after the patient has taken the first plurality of dose units, obtaining a weight of the patient, a hunger level of the patient, or level of side effects caused within the patient by the medication; (3) based upon the weight, hunger level, or level of side effects, adjusting the dose regimen; and (4) providing a second plurality of dose units to the patient according to the adjusted dose regimen. The first plurality of dose units or the second plurality of dose units includes at least one first dose of the medication and at least one second dose of the medication. The first dose is greater than the second dose.

This and other embodiments can include one or more of the following features. The steps can be repeated until a target patient weight is reached. The medication can be phentermine. The second dose can include 0 mg of phentermine. The second dose can include less than 4 mg of phentermine. The first dose can have at least 5 mg more phentermine than the second dose. The first dose can have at least 7.5 mg more phentermine than the second dose. The first dose can include 5-50 mg of phentermine. At least 30% of the first plurality or second plurality of dose units can be second doses. Less than 90% of the first plurality or second plurality of dose units can be second doses. The method can further include weighing the patient before providing the first plurality of dose units. The first or second plurality of dose units can be a weekly supply of dose units. All of the dose units of the first plurality of dose units or the second plurality of dose units can appear identical.

In general, in one embodiment, a method of treating a patient includes: (1) providing a first plurality of dose units of a medication to a patient according to a dose regimen; (2) after the patient has taken the first plurality of dose units, obtaining feedback from the patient regarding symptoms or side effects; (3) based upon the feedback, adjusting the dose regimen; and (4) providing a second plurality of dose units to the patient according to the adjusted dose regimen. The first plurality of dose units or the second plurality of dose units includes at least one first dose of the medication and at least one second dose of the medication. The first dose is greater than the second dose.

This and other embodiments can include one or more of the following features. The medication can be a medication for treating attention deficit disorder. The medication can be an anti-depressant. The medication can be a pain medication. The medication can be a weight-loss medication. The second dose can further include 0 mg of the medication. All of the dose units of the first plurality of dose units or the second plurality of dose units can appear identical.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a table showing the results of various previously published clinical trials using phentermine.

FIG. 1B is a comparison graph showing weight loss of patients in various previously published clinical trials using phentermine.

FIG. 2 is a chart showing exemplary dose pattern levels of phentermine as described herein.

FIG. 2A is chart showing another exemplary set of dose pattern levels of phentermine as described herein.

FIG. 3 is a flow chart showing an exemplary method of inducing weight loss by varying dosages of phentermine as described herein.

FIG. 4 shows an exemplary weekly pack of medication dose units as described herein.

FIG. 5 is a chart comparing results of varying dosages of phentermine as described herein with results from a previously published clinical trial.

FIG. 6 is a graph showing the average weight loss of patients when varying dosages of phentermine as described herein.

### DETAILED DESCRIPTION OF THE INVENTION

In general, described herein is a treatment method that includes providing a patient with a patterned dose regimen of a standard or moderate dose of a medication (e.g., a dose that is traditionally effective when given alone or repetitively) with a low dose of the medication (e.g., a dose that, given by itself or repeatedly, would not provide the desired effects or build tolerance to the medication) to induce a desired effect. For example, the method can include providing a patterned dose regimen that modulates a moderate dose with a low dose to treat weight loss over a period of time, such as greater than twelve weeks. Specifically, the present invention can be used to provide an individual with a phentermine dose regimen that minimizes phentermine habituation and side effects.

Thus, in one embodiment, a method of inducing weight loss in an individual, such as an overweight or obese individual, includes a treatment regimen of varying moderate and low doses. The treatment regimen can include seven days of phentermine dose pattern consisting of moderate doses and low doses in a blinded administration repeated over a treatment period of 12-36 weeks with varied weekly patterns in response to reported patient's hunger, weight loss or gain and side effects. The treatment regimen can be used to achieve substantial weight loss without loss of phentermine's effect and minimization of side effects.

Typical FDA approved moderate marketed therapeutic doses of phentermine in the United States are 37.5 mg and 15 mg per capsule. As used herein, the term low dose can mean any sub-therapeutic dose of a drug which would have little or no clinical effect if given continuously and/or is typically below the FDA minimum recommended dose, including a very low dose such as less than 4 mg, less 2 mg, less than 1 mg, or 0 mg. Further, the moderate dose can include 5 mg-50 mg, such as 7.5 mg-35 mg of phentermine. The moderate dose in each weekly supply can include at least 5 mg, at least 7.5 mg more, or at least 10 mg more phentermine than the low dose.

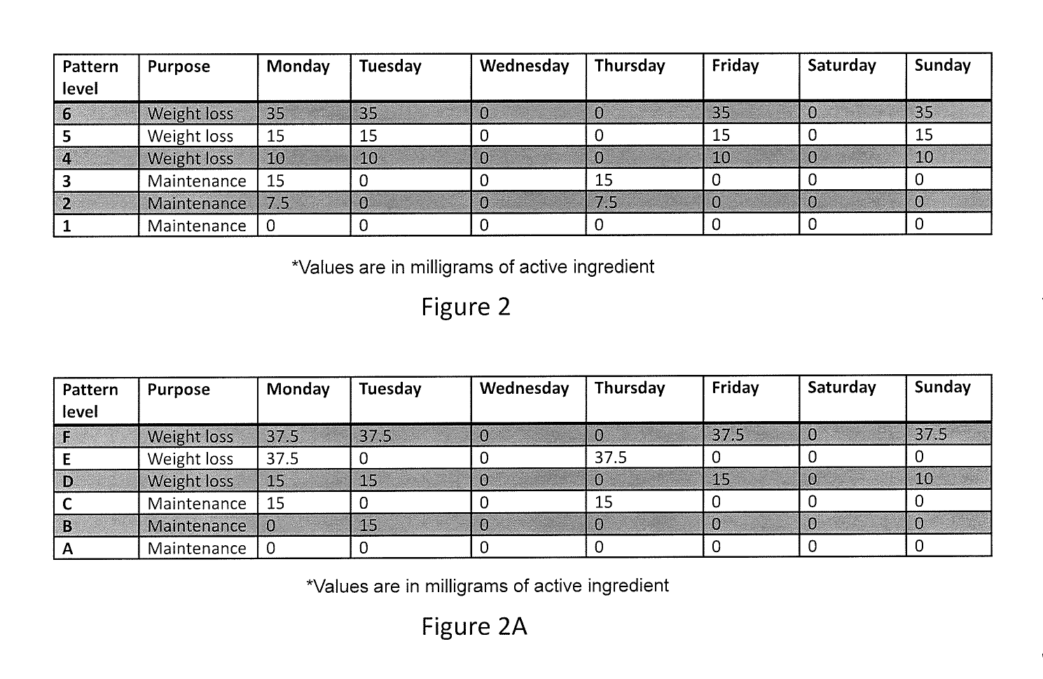

The dose patterns can include a variation of moderate and low doses. For example, in a one-week regimen, at least 25% or at least 30% of the prescribed doses can be low doses. If a single dose is given each day for a week, at least one, at least two, or at least three of the seven doses can be the low dose. Further, at least one dose, at least two doses, or at least three doses in the one-day for a week regimen can be the moderate dose. Two exemplary sets of dose regimens of phentermine are shown in FIGS. 2 and 2A. Referring to FIG. 2, the various regimen options can vary from pattern level 1 with the least amount of medication (all low doses) up to pattern 6 with the greatest amount of medication (four moderate doses of 35 mg each). Likewise, referring to FIG. 2A, the various regiment options can vary from pattern level A with the least amount of medication up to pattern F with the greatest amount of medication. In some embodiments, 30%-90% of the doses in a single week can be low doses.

In some embodiments, additional dose regimens can be provided to the patient based upon feedback or measurements taken from the patient after the patient has been administered a first dose regimen. For example, the patient's hunger level and/or level of side effects and/or weight can be used to determine the patient's next dose regimen. A patient's exercise level and/or amount of sleep can also be used as an input to determine the proper dose pattern level.

An exemplary method for treating a patient with phentermine to induce weight loss can thus include:

(i) Weighing the patient;

(ii) Administering one pill a day of either a moderate dose of phentermine or a low dose of phentermine for a week in one of several weekly dose patterns, such as the patterns described in FIG. 2;

(iii) Weighing the patient during or after the weekly regimen and/or monitoring the patient's hunger and/or side effects; and

(iv) Based on the weight change, hunger level, and/or side effects reported, adjusting the dose pattern level taken for the next week; and

(v) Repeating steps (i-iv) until the patient loses their target excess body weight.

The type of dose regimen (e.g., dose patterns levels 1 to 6 in FIG. 2 or pattern levels A-F in FIG. 2A) can be selected for a patient for a given time period (e.g., week) using an algorithm that is designed to minimize patient exposure to the drug, minimize side effects, and induce the desired weight loss. For example, in one embodiment, if significant side effects are reported, then the dose pattern level can be reduced. If hunger persists, but little to no side effects are reported, then the dose pattern level can be increased. If the weight is not reducing by a goal amount (e.g., 1 lb/week, 1.51 bs/week, 2 lbs/week), then the dose pattern level can be increased. If the weight is reducing by the goal amount per week, then the dose pattern level can be kept constant. In some embodiments, a patient can receive less than 50%, such as less than 40% or approximately 25% of the amount of medication using this algorithm that he or she would have on a standard continuous dose.

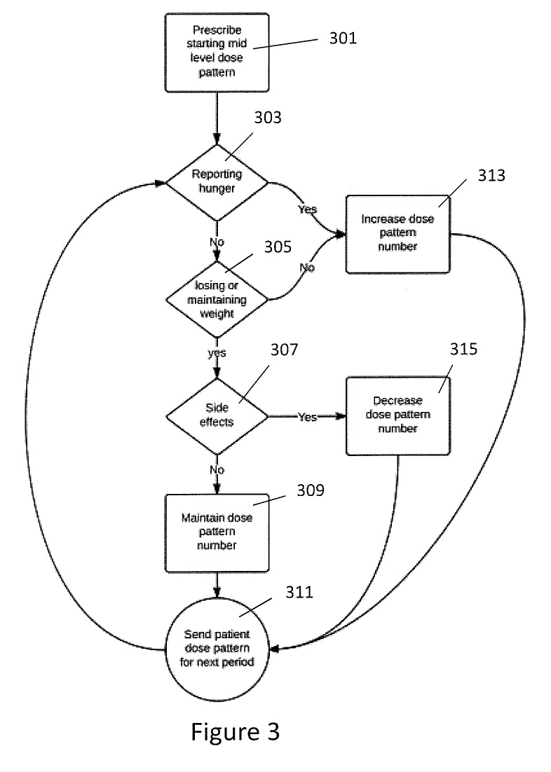

An exemplary method 300 of treating a patient using such an algorithm is shown in FIG. 3. At step 301, a prescribed dose pattern is given to the patient. At step 303, the patient reports his or her hunger. If the patient reports increased or standard hunger levels, then the overall dose or dose pattern level is increased at step 313. On the other hand, if the patient reports decreased hunger levels, then it can be determined at step 305 whether the patent is losing or maintaining weight. If not, then at step 313, the dose or dose pattern level is increased. However, if the patient has lost weight, then it can be determined if there are substantial side effects associated with the dose at step 307. If there are substantial side effects, then the dose or dose pattern level can be decreased. If there are not substantial side effects, then the dose pattern level can be maintained at step 309. Once the next dosage has been determined (either at steps 313, 315, or 309), then the new dose pattern can be provided to the patient at step 311. The method 300 can be performed at any desired time. For example, the method might be performed one a week, once every two weeks, once a month, or even once every day or two.

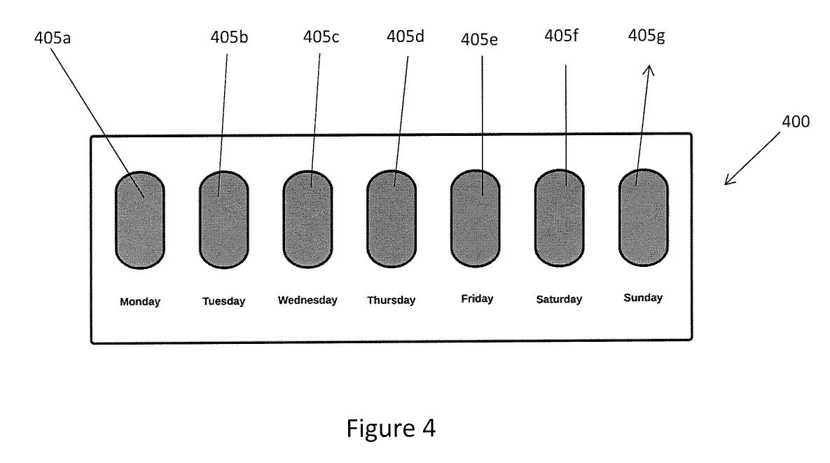

In some embodiments, referring to FIG. 4, the sets of dose regimens can be provided to the patient in a single pack, such as a blister pack. For example, there can be seven dose units 405a-g (e.g. pills, capsules, or vials) that appear identical (i.e., the user cannot tell the difference between the different dose amounts) in a single pack 400. Because the dose units 105a-g appear identical, but actually may include different doses of medication (e.g., according to the dose pattern levels shown in FIGS. 2 and 2A), the various dose units can be marked to indicate when the patient should take each particular dose unit. For example, the dose units can be marked with the days of the week, as shown in FIG. 4. Alternatively or in addition, the dose units 405a-g can be numbered. The pack can include a weekly, biweekly, or monthly supply of medication.

Additional objectives, advantages, and novel features of the present invention will become apparent to one ordinarily skilled in the art upon examination of the following example, which is not intended to be limiting.

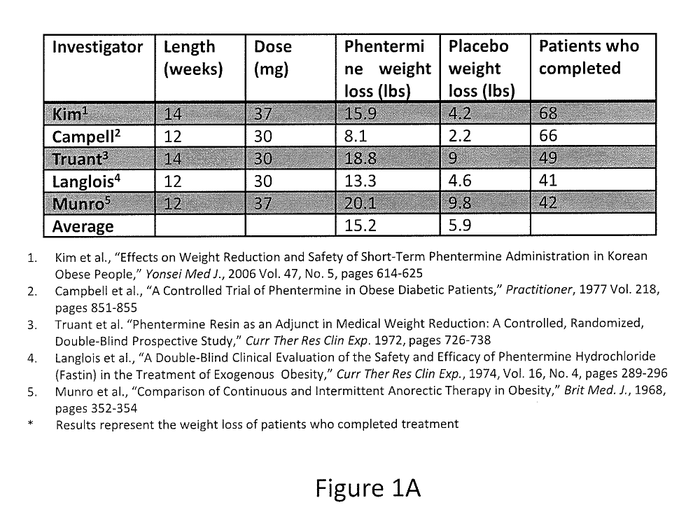

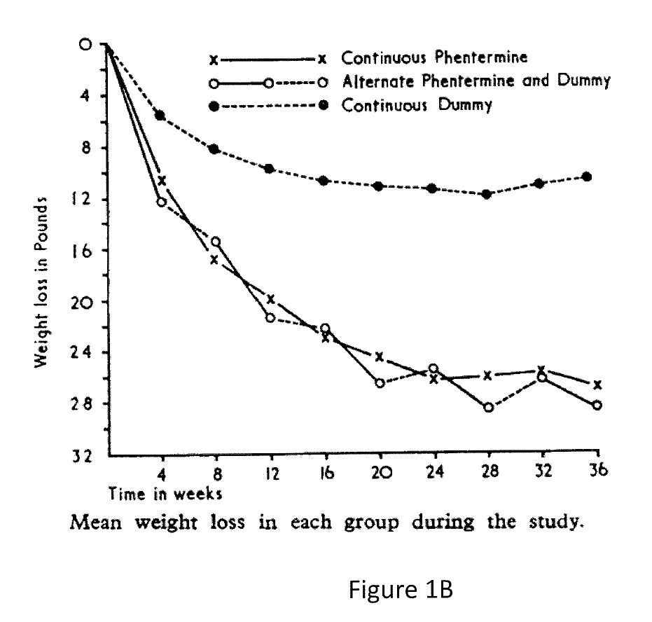

A study was performed including 23 overweight (7) and obese (16) patients whose average initial weight was 207 lb. Inclusion and exclusion criteria were similar to those of many published phentermine clinical trials. The patients were offered conventional dietary advice, provided meal replacements in the form of one to two commercial shakes (Usana Health Sciences, Salt Lake City, Utah), and were encouraged to exercise. Historical controls available from many published data about weight loss that involved dietary advice, meal replacement, and encouragement to exercise were used. An example of the results from published studies using such controls is shown in FIG. 1A.

During the study, each patient was prescribed a customized dose pattern level of moderate capsules of phentermine and low dose capsules of phentermine that were provided on a biweekly basis in a 7-compartment blister pack where each blister contained a daily red capsule. The patient was instructed to take the appropriate daily capsule in the morning around breakfast time. The pack was labeled “each capsule contains up to 35 mg of phentermine.” In this example, each capsule contained 35 mg, 15 mg, 10 mg, 7.5 mg or 0 mg of phentermine in addition to inert excipients. The patients received two dose pattern weekly medications strips every two weeks.

In addition to the pills, patients were given a smartphone app or access to a browser-based reporting form where they could report their daily weight, drug compliance, hunger level, exercise level, and side effects experienced. The data was fed directly to a database, which was analyzed by the physician administering the trial and his assistant.

The algorithm for treatment included the following steps:

(1) Each patient was started on a weight loss dose pattern with a midlevel weekly phentermine dose pattern level 2 as shown in FIG. 2. Patients with concerns about high levels of sensitivity to medicines in the past were started on the lowest effective phentermine dose.

(2) The maximum tolerated weight loss dose pattern for the patient was determined by reviewing the patient's current dose level pattern and his or her electronically reported daily hunger level and side effects such as sleeplessness.

(3) If the patient had significant side effects such as insomnia or nervousness, his or her dose pattern level was reduced, for example from 3 to 2 or from 4 to 3.

(4) If the patient reported persistent hunger but no significant side effects, his or her dose pattern level was increased, for example from 3 to 4 or from 4 to 5.

(5) If the patient's weight was dropping at least 1.5 lbs/week or 6 lbs/month, his or her dose pattern level was kept constant until they achieved their target weight.

(6) If the patient's weight failed to drop by at least 1 lbs/week or 6 lbs/month and hunger was limiting their progress, his or her dose pattern was increased for the subsequent week unless limited by adverse effects or sensitivity to medicine.

(7) Once the patient achieved his or her target, the patient was put on a maintenance dose level patterns comprising of the lowest dose level pattern used in their treatment.

(8) After 12 weeks of the maintenance dose pattern level, patients were generally adjusted to the lowest dose pattern level. If a patient started to gain weight while on the maintenance dose pattern level, it was increased for the subsequent period, for example from 1 to 2 or 2 to 3, etc., and the process was restarted at step 5 or 6.

(9) All therapy was discontinued after 9 months or earlier if the patient displayed stable weight for more than 3 months and reported no hunger issues.

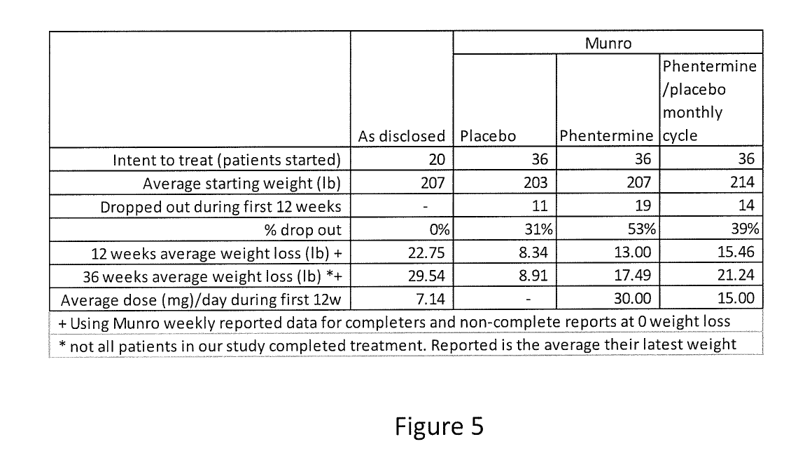

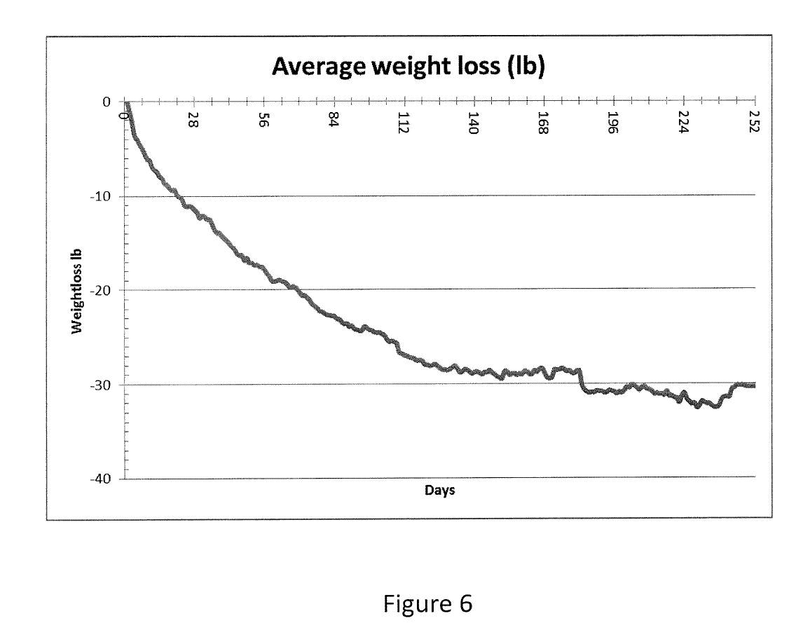

A graph of the results from the study is shown in FIG. 6. Further, a comparison between the results achieved by the present study and those reported by Munro is provided in FIG. 5.

The hunger and weight responsive patient-specific regimen of the study allowed patients to achieve substantial weight loss and sustain it over long intervals exceeding average of 31 treatment weeks. The average twelve-week weight loss was 23 lb. This was achieved for the group on intent to treat basis compared with 15 lbs. reported by Munro with alternating monthly placebo/active trial (see FIG. 5).

The drug treatment was stopped when the patient indicated that they felt like they could manage hunger without the drug. The average period of drug treatment was 23 weeks. None of the patients failed to complete the first 12 weeks in the program, which indicates high level of tolerability of the regimen. Lower reported side effects rate was observed once the initial dose was titrated.

The average total drug exposure was 75% lower than a typical phentermine regimen and 50% lower than Munro monthly phentermine/placebo group. Average period of therapy was 31 weeks. The trial is ongoing, and each patient was offered to continue for 36 weeks (9 months).

Historically, about one-fourth of the patients stay at a constant weight after completing phentermine weight loss therapy that is limited to a maximum continuous effective dosing of 12 weeks by the FDA. The alternating moderate and low doses in weekly patterns described herein allows the clinician to administer a long-term drug regimen with beneficial results because the patient continues to achieve steady control of hunger without building tolerance to the drug. The data above demonstrates that patients are able to lose more weight than in previous phentermine trials and maintain that weight loss for a longer period. This increases the likelihood of permanent weight loss and long-term behavioral changes. The inventors demonstrated that the combination of phentermine with low dose is both safe and effective as a treatment of overweight and obesity.

Although described above using treatment of weight loss with phentermine as an example, it is to be understood that other medications can be used and/or other indications can be treated using the methods, systems, and kits described herein. For example, other obesity drugs, such as a combination of phentermine and torpiramate, could be used. Alternatively, the methods and systems described herein can be used to treat attention deficit disorder (e.g. with a psychostimulant or stimulant), mental disorders, such as depression (e.g., with an anti-depressant), and/or pain (e.g. with a narcotic or neuropathic). Advantageously, the patient can provide feedback on symptoms (e.g. hyperactivity or mood) and side effects and then be provided medication in dose pattern levels as described above based upon the feedback. The dose schedule can be chosen that administers the minimum effective active dose and extends the interceding number of days that the patient receives a low dose while achieving the clinical end points. The methodologies described herein can be particularly advantageous when using medications that commonly cause adverse side effects or engenders drug tolerance when used continuously.

Although described above as modulating moderate and low doses in the dose patterns, in some embodiments, the moderate dose can be provided and then no dose unit can be provided in place of the low dose.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of promoting weight loss in a patient using a drug, where the drug comprises an approved moderate marketed dose amount having a continuous administration period limit, the method comprising:
providing a dose pattern of the drug to the patient, where the dose pattern comprises a dosage amount of the drug distributed in a plurality of dose units each having a varying amount of the drug, where at least one dose unit comprises a low dose of the drug that is below the approved moderate marketed dose amount;
instructing the patient to administer the dose pattern over a period of time;
receiving a feedback from the patient after the period of time, where the feedback comprises an effect of the dose pattern on the patient;
analyzing the feedback to revise the dose pattern, wherein analyzing the feedback comprises:
reducing the dosage amount of the dose pattern if the feedback includes an adverse side effect of the drug;
increasing the dosage amount of the dose pattern if the feedback indicates a hunger level is not decreasing or if the feedback indicates a patient's weight is not decreasing or not maintained;
maintaining the dosage amount of the dose pattern if the feedback indicates the hunger level decreases, the patient's weight is decreasing or maintained, and a lack of the side effect of the drug; and
providing a revised dose pattern of the drug to the patient and repeating the steps such that the dosage amount of the dose pattern is minimized to prevent building the patient's tolerance to the drug while extending weight loss to an effective period that is greater than the continuous administration period limit.

2. The method of claim 1, where the low dose comprises an amount of the drug that would not produce a weight loss effect if given repeatedly.

3. The method of claim 1, where the plurality of dose units in the pattern comprise at least one moderate marketed dose amount of the drug and at least one low dose of the drug.

4. The method of claim 1, where repeating the steps occurs until the patient reaches a target weight.

5. The method of claim 1, where all of the dose units appear identical.

6. The method of claim 1, where receiving the feedback from the patient comprises receiving the feedback using a smartphone app or a browser-based reporting form.

7. The method of claim 6, where receiving the feedback occurs daily.

8. The method of claim 1, where the effect is selected from a group consisting of a side effect of the drug, a hunger level, a patient's weight.

9. The method of claim 1, where the drug comprises Phentermine.

10. A method of promoting weight loss in a patient using a drug, where the drug comprises an approved moderate marketed dose amount having a continuous administration period limit associated with the approved moderate marketed dose amount, the method comprising:
providing a dose pattern of the drug to the patient, where the dose pattern comprises a dosage amount of the drug distributed in a plurality of dose units each having a varying amount of the drug, where at least one dose unit comprises a low dose of the drug that is below the approved moderate marketed dose amount;
instructing the patient to administer the dose pattern over a period of time;
monitoring the patient to produce a minimized effective active dose pattern to prevent a side effect of the drug and maintain efficiency of the drug results while minimizing the dosage amount by providing dose units comprising both the low dose with the approved moderate marketed dose amounts; and
providing the minimized effective active dose pattern to the patient and repeating the steps, where the minimized effective active dose pattern minimizes the patient's exposure to the drug to prevent the patient from building a tolerance to the drug and allows the patient to continue the drug for a period beyond beyond the continuous administration period while losing or maintaining weight.

11. The method of claim 10, where the low dose comprises an amount of the drug that would not produce a weight loss effect if given repeatedly.

12. The method of claim 10, where the plurality of dose units in the dose pattern comprise at least one moderate marketed dose amount of the drug and at least one low dose of the drug.

13. The method of claim 10, where repeating the steps occurs until the patient reaches a target weight.

14. The method of claim 10, where all of the dose units appear identical.

15. The method of claim 10, where the dose units in the dose pattern and the dose units in the minimized effective active dose pattern appear identical.

16. The method of claim 10, where monitoring the patient comprises receiving a feedback from the patient using a smartphone app or a browser-based reporting form.

17. The method of claim 16, where receiving the feedback occurs daily.

18. The method of claim 10, where monitoring the patient comprises monitoring the patient for an effect selected from a group consisting of an adverse side effect of the drug, a hunger level, a patient's weight.

19. The method of claim 10, where the drug comprises Phentermine.

20. A method of promoting weight loss in a patient using a drug having an approved moderate marketed dose amount, the method comprising:
providing a dose pattern of the drug to the patient, where the dose pattern comprises a dosage amount of the drug distributed in a plurality of dose units, where the plurality of dose units include at least one approved moderate marketed dose amount and a low dose amount that would not produce a weight loss effect if given repeatedly;
instructing the patient to administer the dose pattern over a period of time;
receiving feedback from the patient electronically;
analyzing the feedback from the patient to adjust the dose pattern to obtain a minimized effective active dose pattern that prevents a side effect of the drug and maintain efficiency of the drug results while minimizing the dosage; and
providing the minimized effective active dose pattern to the patient and repeating the steps over an effective period of time, where the minimized effective active dose pattern minimizes the patient's exposure to the drug to prevent the patient from building a tolerance to the drug over the effective period of time and allows the patient to continue using the drug for the effective period of time, where the effective period of time is greater than a continuous administration period associated with the approved moderate marketed dose amount.

21.-27. (canceled)
